(19) United States
(12) Patent Application Publication
(10) Pub. No.: US 2026/0124048 A1
(43) Pub. Date: May 7, 2026

(54) METHOD FOR CONTROLLING A TRANSTIBIAL PROSTHESIS AND TRANSTIBIAL PROSTHESIS

(71) Applicant: Otto Bock Healthcare Products GmbH, Vienna (AT)

(72) Inventors: Andreas Bohland, Vienna (AT); Alexander Pappe, Vienna (AT); Martin Seyr, Vienna (AT)

(21) Appl. No.: 19/119,311

(22) PCT Filed: Oct. 5, 2023
(86) PCT No.: PCT/EP2023/077557

§ 371 Date: Apr. 8, 2025

(30) Foreign Application Priority Data
Oct. 10, 2022 (DE) 10 2022 126 222.7

(51) Int. Cl. A61F 2/66; A61F 2/70; A61F 2/76

(52) U.S. Cl. CPC A61F 2/6607; A61F 2/70; A61F 2/76; A61F 2002/7625

(57) ABSTRACT

The invention relates to a method for controlling a lower leg prosthesis comprising a foot element, a lower leg element pivotably arranged thereon and an adjustable resistance device for applying a resistance against a swivelling of the foot element relative to the lower leg element, wherein
a first switching angle is defined as a predetermined ankle angle value between the lower leg element and the foot element,
a second switching angle is defined as a predetermined lower leg angle value of the absolute angle of the lower leg,

the method comprising the steps:
at least also on the basis of a height difference between the position of the foot element in a step cycle and the position of the foot element in the previous step cycle, determining whether the wearer of the lower leg prosthesis is walking downhill, and
increasing the resistance of the resistance device to a predetermined resistance value when a block criterion is met,

wherein the block criterion is met

if it has been determined that the wearer is not walking downhill and the second switching angle is reached, or

if it has been determined that the wearer is walking downhill and the switching angle reached later in the step cycle is reached.

The invention relates to a method for controlling a lower leg prosthesis comprising a foot element, a lower leg element pivotably arranged thereon and an adjustable resistance device for applying a resistance against a swivelling of the foot element relative to the lower leg element. The invention also relates to a lower leg prosthesis that can be controlled using such a method.

Lower leg prostheses of the type described above can generate different resistances against a swivelling of the foot element relative to the lower leg element during a step cycle via the adjustable resistance device. This has been used in the prior art for many years to imitate the natural gait. Generally, it is important that the resistance is significantly increased approximately in the middle of the stance phase of a step cycle. This often goes so far that further swivelling of the foot element relative to the lower leg element is no longer possible from this point onwards. The stance phase of a step cycle is defined by the foot element being in contact with the ground.

From the moment the resistance is increased, a further swivelling of the foot element relative to the lower leg element is not possible or only possible with difficulty. The foot then rolls over the forefoot, which is essentially formed by the toes. This changes the lever length over which rolling takes place.

From the prior art, it is known to make the point at which the resistance is increased dependent on the gradient of the surface. For example, if the wearer walks up a ramp, the switching point at which resistance is increased should be postponed, i.e. shifted to a later point in time, within the step cycle.

In the process, a foot element and/or a lower leg element is often used that is equipped with an absolute angle sensor with which the inclination of the foot element during the stance phase of the step cycle can be determined. This renders it easy to identify, for example, whether the wearer is walking up or down a slanted plane, such as a ramp. However, this method cannot be applied if, for example, it is to be determined whether the wearer of the lower leg prosthesis is walking up or down stairs. The angle of inclination of the foot element is the same regardless of the movement of direction as the step is horizontal. In addition, the method described reaches its limits when the terrain on which the wearer of the lower leg prosthesis is moving does not form an even surface. If the surface is uneven, the inclination of the foot element cannot or at least cannot always be used to reliably determine the gradient of the surface and when the switching point should occur.

As such, it is known from DE 10 2012 125 256 A1 to define two switching angles. The first switching angle is defined as a predetermined ankle angle value between the lower leg element and the foot element. Therefore, as soon as the ankle angle that is detected to control the lower leg prosthesis reaches said predetermined ankle angle value, the first switching angle is deemed to have been reached. The second switching angle is defined as a predetermined lower leg angle value of the absolute angle of the lower leg. Regardless of the value of the ankle angle between the lower leg element and the foot element, the second switching angle is deemed reached when the absolute angle of the lower leg reaches the predetermined lower leg angle value. In order to now control the lower leg prosthesis, it must be determined which of the two defined switching angles is to be the angle at which the resistance of the resistance device should be increased. The prior art suggests always using the switching angle that occurs first within the respective step cycle, regardless of a detected position of the foot element in the stance phase.

This is a good choice in many situations, but in some situations it does lead to problems. For example, the switching angle may be defined as a right angle, i.e. a 90° angle. This means that there is a right angle between the lower leg element and the foot element, for example the contact surface that comes into contact with the ground, when the first switching angle is defined. The second switching angle may also be defined as a 90° angle. This means that the second angle is reached when the lower leg element is perpendicular, i.e. extends parallel to the acting force of gravity.

These definitions of the two switching angles mean that the second switching angle is reached first when walking downwards along a ramp. Consequently, the resistance is increased when the lower leg element is vertical, which leads to a more stable stance, but results in an unnatural gait. However, this definition of the two switching angles means that the first switching angle is reached first when walking up the ramp. This, however, is too early when walking upwards and leads to a premature increase in the resistance applied by the resistance device.

The invention thus aims to further develop a method for controlling a lower leg prosthesis in such a way that it makes walking secure and comfortable.

The invention solves the task addressed by way of a method for controlling a lower leg prosthesis comprising a foot element, a lower leg element pivotably arranged thereon and an adjustable resistance device for applying a resistance against a swivelling of the foot element relative to the lower leg element, wherein a first switching angle is defined as a predetermined ankle angle value between the lower leg element and the foot element and a second switching angle is defined as a predetermined lower leg angle value of the absolute angle of the lower leg. According to the invention, the method includes determining, at least also on the basis of a height difference between the position of the foot element in a step cycle and the position of the foot element in the previous step cycle, whether the wearer of the lower leg prosthesis is walking downhill and increasing the resistance of the resistance device to a predetermined resistance if a block criterion is met. According to invention, the block criterion is met if it has been determined that the wearer is not walking downhill and the second switching angle is reached, or if it has been determined that the wearer is walking downhill and the switching angle reached later in the step cycle is reached.

Unlike in the prior art, the method according to the invention does not require the inclination of the foot element in the stance phase of the step cycle. Instead, it is determined whether the wearer is walking downhill, wherein the local inclination of the surface, which is decisive for the inclination of the foot element in the stance phase, is not taken into account. If the determination shows that the wearer is not walking downhill, the resistance of the resistance device is increased when the second switching angle is reached. This is the case when the absolute angle of the lower leg reaches the predetermined lower leg angle value. The increase in resistance in this case is therefore completely independent of the local inclination of the ground and therefore also the inclination of the foot element in the stance phase. If, however, the determination shows that the wearer is walking downhill, the block criterion is met when the switching angle later in the step cycle is reached.

This results in the most natural gait possible when it is correctly recognized whether the wearer of the lower leg prosthesis is walking downhill or not. However, methods for controlling a lower leg prosthesis should preferably ensure secure operation of the lower leg prosthesis, even in the event of incorrect detection or determination, as the wearer of the lower leg prosthesis could otherwise fall and hurt themselves. Consequently, if during the method according to the invention it is incorrectly detected that the wearer is walking downhill, the switching angle reached later in the step cycle is used to increase the resistance of the resistance device. Since the wearer is actually not walking downhill in this case but, for example, uphill, this is the second switching angle, which is defined as the predetermined lower leg angle value. Consequently, the correct switching angle is used, even in the event of a false detection of downhill walking.

Conversely, if the wearer is walking downhill and this is not recognized, the second switching angle is used as a block criterion. The resistance is therefore increased when the absolute angle of the lower leg reaches the predetermined lower leg value. However, this is usually the switching angle that is reached earlier in a step cycle when walking downhill and thus the wrong switching angle for a wearer walking downhill. This does not result in an optimal gait, but does enable a stable stance and consequently does not constitute a significant safety issue. The effect of this incorrect control on the wearer is relatively unproblematic, especially for a foot with a carbon spring, as the carbon spring allows the wearer to roll over the forefoot and thus to brake.

Preferably, the predetermined resistance value is big enough to completely prevent a further swivelling of the foot element relative to the lower leg element.

Preferably, the height difference between the position of the foot element in the stance phase of the two step cycles is determined. The position of a foot element changes over the course of the stance phase not only in the forward direction, but also in a direction perpendicular to it. At the end of the stance phase, the foot is raised and only placed back on the ground again at the start of the new stance phase. If a meaningful height difference is to be determined, it is advantageous to use the same point in time within the two consecutive step cycles in order to determine the position of the foot at this particular point in time and to be able to calculate a difference. The position is preferably determined during the stance phase, wherein determination of the position in the stance phase is particularly advantageous as it does not change over a longer period of time, namely the duration of the stance phase, and can thus be very precisely determined.

In one preferred embodiment, however, the height difference is not used directly to determine whether the wearer of the prosthesis is walking up-or downhill. Rather, a pitch angle is preferably calculated that is determined from the height difference and a difference in length, which has likewise been determined from the difference of the positions of the foot element at a particular point in time within two consecutive step cycles. This results in a gradient triangle, the pitch angle of which can be easily calculated. This pitch angle is preferably used as criterion in order to determine whether the wearer of the prosthesis is walking up-or downhill. In one preferred embodiment, the pitch angle is compared with a predetermined limit angle. If the pitch angle is above the predetermined limit angle, the wearer of the lower leg prosthesis is not deemed to be walking downhill. However, if the pitch angle is below the predetermined limit angle, the wearer is deemed to be walking downhill.

The limit angle is preferably between 0° and −10°, especially preferably −3°.

The predetermined ankle angle value is preferably between 80° and 100°, especially preferably 90°. The predetermined lower leg angle value is preferably between 80° and 100°, especially preferably 90°. Preferably, the predetermined ankle angle value is amended by an angle of correction resulting from the heel height of a shoe worn by the wearer. The higher the heel of the shoe, the larger the predetermined ankle angle value. The greater the heel height of the shoe, the greater the shift in the neutral position of the ankle, i.e. the angle between the foot element and the lower leg element during relaxed standing. This change in the neutral position is taken into account by the correction angle.

The heel height is dependent on the length of the foot. For example, a foot length of 22 cm and a heel height of 3 cm would result in an angle of approximately 8°. The measured angle or alternatively the predetermined ankle angle value must be corrected by this angle, which forms the correction angle, in the sense of an offset.

The invention also solves the task addressed by way of a lower leg prosthesis with a foot element, a lower leg element pivotably arranged thereon and an adjustable resistance device for applying a resistance against a swivelling of the foot element relative to the lower leg element, wherein the lower leg prosthesis comprises at least one sensor for determining a height difference, at least one sensor for determining an ankle angle, at least one sensor for determining the absolute angle of the lower leg, and an electrical control unit that is configured to conduct a method according to one of the preceding embodiments.

The sensor for determining the absolute angle of the lower leg element is preferably a so-called IMU (inertial measurement unit), for example a combination of accelerometers and/or gyroscopes, with which the orientation of the lower leg element in space is determined and, from this, the absolute angle of the lower leg element is calculated. Alternatively or additionally, the sensor is configured to determine the absolute angle of the foot element and the angle between foot element and lower leg element, so that they can be used to calculate the absolute angle of the lower leg element.

The electrical control unit is preferably an electronic data processing device. Particularly preferably, the at least one sensor for determining the ankle angle comprises at least one absolute angle sensor for determining the absolute angle of the lower leg element and at least one absolute angle sensor for determining the absolute angle of the foot element.

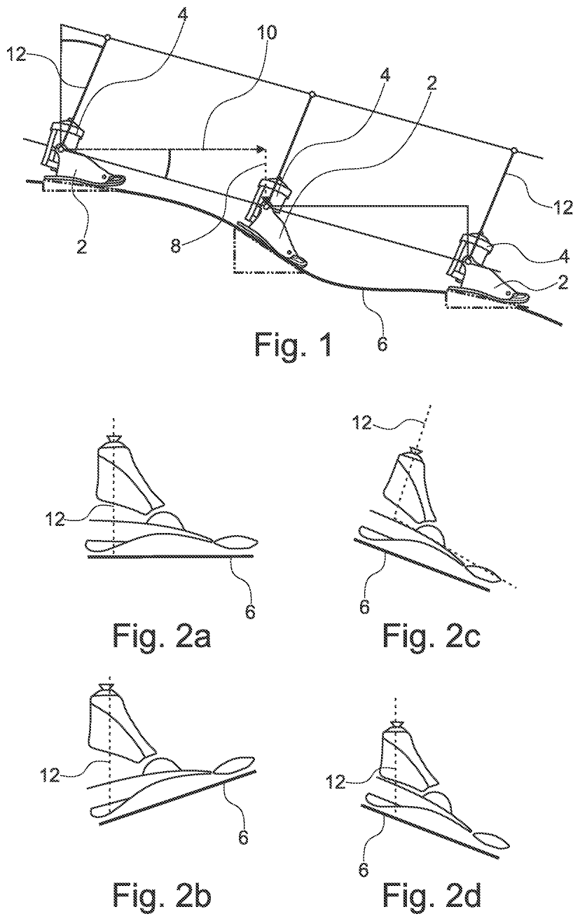

In the following, a number of embodiment examples of the invention will be explained in more detail with the aid of the accompanying diagrams. They show:
FIG. 1—the schematic representation of different variables and

FIG. 2a-2d—reaching the block criterion on different surfaces.

FIG. 1 schematically depicts a section of a lower leg prosthesis with a foot element 2 on which a lower leg element 4 is arranged. The foot element 2 can be swivelled relative to the lower leg element 4. FIG. 1 shows the position of the lower leg prosthesis in three consecutive stance phases, i.e. the stance phases of three consecutive step cycles. In each case, the foot element 2 is in contact with the surface 6. In each case, this surface has a local inclination, which corresponds to the inclination of the foot element 2 at the respective point of the surface 6. This local inclination is illustrated by the triangle depicted below the foot element 2. It does not have to be used to control the lower leg prosthesis and is preferably not used.

Instead, in the embodiment example shown a height different 8 and a length difference 10 are determined, wherein the respective difference between the positions of the foot element 2 between two consecutive steps is calculated. In the embodiment example shown, both are as large between the first position shown and the second position shown as between the second position shown and the third position shown. The global inclination of the surface 6 is therefore constant, although the local inclination can vary greatly and does so in the example shown.

FIG. 1 shows the direction of the lower leg element 4 by way of a line 12. The first switching angle corresponds to a predetermined value of the angle between the line 12 and the local inclination of the surface 12, which corresponds to the direction of the foot element 2. The second switching angle corresponds to the predetermined orientation of the line 12, i.e. a predetermined value of the absolute angle of the lower leg element 4.

In the following, the first switching angle should correspond to an ankle angle value of 90° and the second switching angle should correspond to an absolute angle of 90°, i.e. a vertical alignment of the lower leg element 4.

In FIG. 2 a, the lower leg prosthesis is schematically depicted on an even surface 6. The line 12, which is depicted as a dashed line, is perpendicular on the surface 6, so that the first switching angle is reached. The line 12 is also aligned vertically, so that the second switching angle is also reached. The wearer of the prosthesis is not walking downhill. If this is correctly recognized in the method, the second switching angle is used for control, i.e. the resistance of the resistance device, not depicted, is increased when the lower leg element 4 is perpendicular. If the control unit incorrectly recognized that the wearer is going downhill, the second switching angle reached is used for control. As both switching angles are reached at the same time, there is no problem and the increase in resistance takes place at the right time as expected by the wearer of the prosthesis.

FIG. 2b depicts the situation in which the surface 6 is designed as an upward ramp. Since the wearer is not walking downhill in this case either, the second switching angle is used to control the prosthesis, i.e. the resistance is increased when the absolute angle of the lower leg element 4, i.e. the line 12 in FIG. 2b, is perpendicular. This is shown in FIG. 2b. If it had been incorrectly recognized that the wearer is walking downhill, the control unit would have increased the resistance when the switching angle reached later was reached. In the situation depicted in FIG. 2b, this is the second switching angle, so that in both cases, the increase in resistance occurs at the time expected by the user or wearer of the prosthesis.

FIG. 2c depicts the situation in which the surface 6 is designed as a downward ramp. In this case, the wearer is walking downhill. Therefore, the first switching angle is used to control the prosthesis, i.e. the resistance is increased when the ankle angle between the line 12 of the lower leg element 4 and the foot element 2 is 90°. This is shown in FIG. 2c. If it had been incorrectly recognized that the wearer is not walking downhill, the control unit would have increased the resistance when the second switching angle was reached. The increase in resistance thus occurs when the lower leg element and therefore its line 12 are perpendicular. This is earlier than the wearer expects, but does not mean a risk of falling as the early increase in resistance still ensures a stable stance.

FIG. 2d depicts the situation in which the wearer of the lower leg prosthesis is not walking downhill, but the surface has a local unevenness. In this case, the second switching angle is used to control the prosthesis, which is reached in FIG. 2d.

### REFERENCE LIST

2 foot element
4 lower leg element
6 surface
8 height difference
10 length difference
12 line

1. A method for controlling a lower leg prosthesis comprising a foot element, a lower leg element pivotably arranged thereon and an adjustable resistance device for applying a resistance against a swivelling of the foot element relative to the lower leg element, wherein
a first switching angle is defined as a predetermined ankle angle value between the lower leg element and the foot element,
a second switching angle is defined as a predetermined lower leg angle value of the absolute angle of the lower leg,
the method comprising the steps:
determining, at least also on the basis of a height difference between the position of the foot element in a step cycle and the position of the foot element in the previous step cycle, whether the wearer of the lower leg prosthesis is walking downhill, and
increasing the resistance of the resistance device to a predetermined resistance value when a block criterion is met,
wherein the block criterion is met
if it has been determined that the wearer is not walking downhill and the second switching angle is reached, or
if it has been determined that the wearer is walking downhill and the switching angle reached later in the step cycle is reached.

2. The method according to claim 1, characterized in that the predetermined resistance value is big enough to prevent a further swivelling of the foot element relative to the lower leg element.

3. The method according to claim 1, characterized in that the height difference between the position of the foot element in the stance phase of the two step cycles is determined.

4. The method according to claim 1, characterized in that a pitch angle, which is determined from the height difference and a length difference between the positions of the foot element, is used to determine whether the wearer of the lower leg prosthesis is walking downhill.

5. The method according to claim 4, characterized in that the wearer is deemed to be walking downhill if the pitch angle is smaller than a limit angle, which is preferably between 0° and −10° and particularly preferably is −3°.

6. The method according to claim 1, characterized in that the predetermined ankle angle value is between 80° and 100°, preferably 90°.

7. The method according to claim 1, characterized in that the predetermined lower leg angle value is between 80° and 100°, preferably 90°.

8. A lower leg prosthesis with a foot element, a lower leg element pivotably arranged thereon and an adjustable resistance device for applying a resistance against a swivelling of the foot element relative to the lower leg element, wherein the lower leg prosthesis comprises at least one sensor for determining a height difference, at least one sensor for determining an ankle angle, at least one sensor for determining the absolute angle of the lower leg, and an electrical control unit that is configured to conduct a method according to one claim 1.

9. The lower leg prosthesis according to claim 8, characterized in that the at least one sensor for determining the ankle angle comprises an absolute angle sensor for determining the absolute angle of the lower leg element and an absolute angle sensor for determining the absolute angle of the foot element.
